SEQUENCE LISTING (9 sequences)

SEQ ID NO: 1 - DNA, 93 bp, Artificial Sequence
  Description of Artificial SequenceRestriction Sites
cggccgcacgcgtgagctccgcggttcgaatcccgggattcgaacatcgataaaagatctacgtaggtaaccacgtgcggaccgagcggccgc

SEQ ID NO: 2 - DNA, 18 bp, Artificial Sequence
  Description of Artificial SequencePrimer
ggccgggaacggtgcatt

SEQ ID NO: 3 - DNA, 21 bp, Artificial Sequence
  Description of Artificial SequencePrimer
gggcaagggggtgggcctata

SEQ ID NO: 4 - DNA, 77 bp, Artificial Sequence
  Description of Artificial SequenceRestriction Sites
atcgattgaattccccggggatcctctagagtcgacctgcagaagcttgctctcgagcagcgctgctcgagagatct

SEQ ID NO: 5 - DNA, 8 bp, Artificial Sequence
  Description of Artificial SequenceMutated Kozak Sequence
ccaccatg

SEQ ID NO: 6 - DNA, 24 bp, Artificial Sequence
  Description of Artificial SequenceMutated Oligonucleotide
agctaggcgccaccatgggggtgc

SEQ ID NO: 7 - DNA, 20 bp, Artificial Sequence
  Description of Artificial SequenceRestriction Site
ggcgcccctgcaggacatgt

SEQ ID NO: 8 - DNA, 32 bp, Artificial Sequence
  Description of Artificial SequenceD region of AAV ITR
gcggccgcaggaacccctagtgatggagttgg

SEQ ID NO: 9 - DNA, 26 bp, Artificial Sequence
  Description of Artificial SequenceMutated Oligonucleotide
catcgattgaattccaccatgggggt

(19) United States
(12) Patent Application Publication
(10) Pub. No.: US 2004/0087028 A1
(43) Pub. Date: May 6, 2004

(54) Ecdysone-inducible adeno-associated virus expression vectors

(75) Inventor: Janet Cunningham, Alameda, CA (US)

Correspondence Address: ROBINS & PASTERNAK, 1731 EMBARCADERO ROAD, SUITE 230, PALO ALTO, CA, 94303, US

(73) Assignee: Avigen Inc.

(21) Appl. No.: 10/670,449

(22) Filed: Sep. 24, 2003

Related U.S. Application Data
(63) Continuation of application No. 09/706,580, filed on Nov. 3, 2000, now abandoned.
(60) Provisional application No. 60/164,068, filed on Nov. 5, 1999.

(51) Int. Cl. C12N 15/861; C12N 7/00

(52) U.S. Cl. 435/456; 435/235.1

(57) ABSTRACT

The present invention relates to AAV expression vectors that allow the introduction and regulated expression of heterologous genes into mammalian tissues and cells. In certain embodiments, vectors of the present invention are ecdysone-inducible AAV expression vectors and AAV expression vectors encoding EcR and RXR. In this system, the heterologous gene is “turned off” until an inducer such as pon A is provided to the tissues or cells. The present invention also provides methods of using inducible AAV expression vectors.

### CROSS-REFERENCE TO RELATED APPLICATION

This application is related to provisional patent application serial No. 60/164,068, filed Nov. 5, 1999, from which priority is claimed under 35 USC §119(e)(1) and which is incorporated herein by reference in its entirety.

### FIELD OF THE INVENTION

The present invention relates to inducible adeno-associated virus (AAV) expression vectors. More specifically, the present invention relates to ecdysone-inducible AAV expression vectors and virions comprising the same, which allow controlled expression of transfected or transduced genes in a highly regulatable manner.

### BACKGROUND OF THE INVENTION

### A. Gene Therapy

Scientists are continually discovering genes that are associated with human diseases such as diabetes, hemophilia and cancer. Research efforts have also uncovered genes, such as erythropoietin (which increases red blood cell production), that are not associated with genetic disorders but code for proteins that can be used to treat numerous diseases. However, despite significant progress in the effort to identify and isolate genes, a major obstacle facing the biopharmaceutical industry is how to safely and persistently deliver effective quantities of these genes' products to patients.

Currently, the protein products of these genes are synthesized in cultured bacterial, yeast, insect, mammalian, or other cells and delivered to patients by intravenous injection. Intravenous injection of recombinant proteins has been successful but suffers from several drawbacks. First, patients frequently require multiple injections in a single day in order to maintain the necessary levels of the protein in the blood stream. Even then, the concentration of protein is not maintained at physiological levels—the level of the protein is usually abnormally high immediately following injection and far below optimal levels prior to injection. Second, intravenous delivery often cannot deliver the protein to the target cells, tissues or organs in the body. And, if the protein reaches its target, it is often diluted to non-therapeutic levels. Third, the method is inconvenient and severely restricts the patient's lifestyle. The adverse impact on lifestyle is especially significant when the patient is a child.

These shortcomings have led to the development of gene therapy methods for delivering sustained levels of specific proteins into patients. These methods allow clinicians to introduce DNA coding for a gene of interest directly into a patient (in vivo gene therapy) or into cells isolated from a patient or a donor (ex vivo gene therapy). The introduced DNA then directs the patient's own cells or grafted cells to produce the desired protein product. Gene delivery, therefore, obviates the need for daily injections. Gene therapy may also allow clinicians to select specific organs or cellular targets (e.g., muscle, blood cells, brain cells, etc.) for therapy.

DNA may be introduced into a patient's cells in several ways. There are transfection methods, including chemical methods such as calcium phosphate precipitation and liposome-mediated transfection, and physical methods such as electroporation. Although transfection methods are not suitable for in vivo gene delivery, recombinant viruses may be used for such purposes. Current viral-mediated gene delivery methods employ retrovirus, adenovirus, herpes virus, pox virus, and adenoassociated virus (AAV) vectors.

### B. Adeno-Associated Virus-Mediated Gene Therapy

One viral system that has been used for gene delivery is AAV. AAV is a parvovirus which belongs to the genus Dependovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions which lends itself to production, storage and transportation requirements.

The AAV genome is a linear, single-stranded DNA molecule containing approximately 4681 nucleotides. The AAV genome generally comprises an internal non-repeating genome flanked on each end by inverted terminal repeats (ITRs). The ITRs are approximately 145 base pairs (bp) in length. The ITRs have multiple functions, including as origins of DNA replication and as packaging signals for the viral genome.

The internal non-repeated portion of the genome includes two main open reading frames, for the AAV replication (rep) and capsid (cap) genes. The rep and cap genes code for viral proteins that allow the virus to replicate and package the viral genome into a virion. In particular, a family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus; that is, it requires co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

AAV has been engineered to deliver genes of interest by deleting the internal non-repeating portion of the AAV genome (i.e., rep and cap) and inserting a heterologous gene between the ITRs. The heterologous gene may be linked to a heterologous promoter. Termination signals, such as polyadenylation sites, can also be included.

To produce infectious recombinant AAV (rAAV) containing the heterologous gene, a suitable producer cell line is transfected with an AAV expression vector containing a heterologous gene sandwiched between AAV ITRs. AAV helper functions and accessory functions are then expressed in the producer cell. Once these factors come together, the heterologous gene is replicated and packaged as though it were a wild-type AAV genome, forming a recombinant virion. When a patient's cells are infected (or “transduced”) with the resulting rAAV virions, the heterologous gene enters and is expressed in the patient's cells. Because the patient's cells lack the rep and cap genes and the adenovirus accessory function genes, the rAAV are replication defective; that is, they cannot further replicate and package their genomes. Similarly, without a source of rep and cap genes, wild-type AAV cannot be formed in the patient's cells.

In many circumstances, it is necessary or desirable to restrict expression of the heterologous gene. For example, it may be desirable to restrict expression to certain tissues or to certain times. Systems have been developed that allow regulated expression of heterologous genes in mammalian cells and tissues, but these systems suffer from a number of drawbacks. First, these systems are frequently induced by factors or stimuli (e.g., metal ions, heat shock, growth factors, or steroid hormones) that produce pleiotropic effects; that is, the inducer may, in addition to stimulating expression of the heterologous gene, affect the expression of endogenous genes. Second, currently available systems may have basal rates of transcription that are too high-that is, expression of the heterologous gene can not be turned completely “off.” Third, many currently available systems do not permit sufficient expression of the heterologous gene to achieve a therapeutic effect.

From the foregoing, it will be appreciated that it would be an advancement in the art to provide AAV expression vectors that allow expression of a heterologous gene to be turned on and off. It would be a further advancement in the art to provide such expression vectors that allow regulation of the heterologous gene's expression without affecting the expression of endogenous genes. It would be yet a further advancement if the heterologous gene's basal transcription rate was very low, and if that transcription rate could be induced sufficiently so as to produce a therapeutic effect.

Such AAV expression vectors and methods of their use are disclosed herein.

### SUMMARY OF THE INVENTION

The present invention relates to AAV expression vectors and methods of using such vectors. In certain embodiments, a vector of the present invention is an ecdysone-inducible AAV expression vector.

An ecdysone-inducible AAV expression vector of the present invention may be used in conjunction with AAV vectors that encode the ecdysone receptor (EcR). In certain embodiments, AAV vectors that encode a retinoid-X-receptor (RXR) may also be used in conjunction with ecdysone-inducible AAV expression vectors. An inducer such as the insect hormone ecdysone or its analog ponasterone A may be used with the AAV expression vectors of the present invention.

Recombinant AAV virions engineered to carry the expression vectors of the present invention may be used to introduce genetic material into animals, including humans, or isolated animal cells for a variety of research and therapeutic uses. For example, rAAV virions produced using the methods of the present invention may be used to express a protein in animals to gather preclinical data or to screen for potential drug candidates. Alternatively, the rAAV virions may be used to transfer genetic material into a human to cure a genetic defect or to effect a desired treatment.

These and other advantages of the present invention will become apparent upon reference to the accompanying drawing and upon reading the following detailed description.

### BRIEF DESCRIPTION OF THE FIGURES

FIGS 1A-1B schematically depict the regulation of transcription using an ecdysone-inducible mammalian expression system available from Stratagene (La Jolla, Calif.). The synthetic receptor VgEcR is a fusion of the ligand-binding and dimerization domain of the Drosophila ecdysone receptor (EcR), the DNA-binding domain of the glucocorticoid receptor (GR), and the transcriptional activation domain of HSV VP16. FIG. 1A shows that VgEcR and RXR bind as a heterodimer to five copies of the E/GRE recognition sequence (E/GRE×5), which are located upstream of a minimal promoter composed of three SP1 binding sites (SP1×3) and the ΔHsp minimal promoter. The E/GRE recognition sequence consists of inverted half-site recognition elements for the RXR and the GR DNA-binding domains (which are separated by one nucleotide). In the absence of pon A, the inducer, the promoter is tightly repressed by co-repressors. FIG. 1B shows that when pon A binds to VgEcR, the co-repressors are released, coactivators are recruited, and the complex becomes transcriptionally active.

FIG. 2 illustrates the steps taken to generate the pAAV-Ecd1a-hEpo and pAAV-Ecd1b-hEpo plasmids, described in the examples.

FIG. 3 provides restriction maps of pAAV-Ecd1a-hepo and pAAV-Ecd1b-hEpo.

FIGS. 4A-4C are graphs representing the extent of pon A induction of ER-293 cells transfected with AAV vectors according to the invention at various times. FIG. 4A shows the extent of induction at 24 hours; FIG. 4B shows the extent of induction at 48 hours; FIG. 4C shows the extent of induction at 72 hours.

FIGS. 5A-5D are graphs representing the extent of pon A induction of ER-293 cells transduced with recombinant AAV virions comprising AAV vectors of the invention at various times. FIG. 5A shows the extent of induction after 24 hours; FIG. 5B shows the extent of induction after 48 hours; FIG. 5C shows the extent of induction after 72 hours; FIG. 5D shows the extent of induction after 96 hours.

FIG. 6 illustrates the steps taken to generate the pAAV-CMV-EcR plasmid.

FIG. 7 illustrates the steps taken to generate the pAAV-CMV-RXR plasmid.

FIGS. 8A-8C are graphs representing the extent of pon A induction of transfected myotubes at various times. FIG. 8A shows the extent of induction after 24 hours; FIG. 8B shows the extent of induction after 48 hours; FIG. 8C shows the extent of induction after 72 hours.

FIGS. 9A-9D depict AAV expression vectors described in the examples. FIG. 9A shows AAV-Ecd1a-hEpo; FIG. 9B shows AAV-Ecd1b-hEpo; FIG. 9C shows AAV-CMV-EcR; FIG. 9D shows AAV-CMV-RXR.

FIG. 10 shows in vivo induction of Epo expression in mice using the system of the invention. Results using triple vector-injected mice which were also administered pon A are represented by solid diamonds. Results from triple vector-injected mice which were not administered pon A are shown as solid triangles. Results from double vector-injected mice which were also given pon A are shown as open circles.

### DETAILED DESCRIPTION OF THE INVENTION

The practice of the present invention will employ, unless otherwise indicated, conventional methods of chemistry, biochemistry, recombinant DNA techniques and immunology, within the skill of the art. Such techniques are explained fully in the literature. See, e.g., Sambrook, et al., Molecular Cloning: A Laboratory Manual (2nd Edition); Methods In Enzymology (S. Colowick and N. Kaplan eds., Academic Press, Inc.); DNA Cloning, Vols. I and II (D. N. Glover ed.); Oligonucleotide Synthesis (M. J. Gait ed.); Nucleic Acid Hybridization (B. D. Hames & S. J. Higgins eds.); Animal Cell Culture (R. K. Freshney ed.); Perbal, B., A Practical Guide to Molecular Cloning.

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “an antigen” includes a mixture of two or more antigens, and the like.

### I. Definitions

In describing the present invention, the following terms will be employed, and are intended to be defined as indicated below.

By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

By an “AAV vector” is meant a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

“AAV helper functions” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

The term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al., (1989) J. Virol. 63:3822-3828; and McCarty et al., (1991) J. Virol. 65:2936-2945. A number of other vectors have been described which encode Rep and/or Cap expression products. See, e.g., U.S. Pat. Nos. 6,001,650 and 6,027,931, incorporated by reference herein in their entireties.

The term “accessory functions” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

For example, accessory functions can be provided by delivery of helper viruses such as an adenoviruses, a herpesviruses, a cytomegalovirus or a vaccinia virus. Alternatively, elements from these viruses which are involved in AAV replication may be delivered. Adenovirus-derived accessory functions have been widely studied, and a number of adenovirus genes involved in accessory functions have been identified and partially characterized. See, e.g., Carter, B. J. (1990) “Adeno-Associated Virus Helper Functions,” in CRC Handbook of Parvoviruses, vol. I (P. Tijssen, ed.), and Muzyczka, N., (1992) Curr. Topics. Microbiol. and Immun. 158:97-129. Specifically, early adenoviral gene regions E1A; the E1B 19 kDa protein coding region; the E2A 72 kDa protein coding region; open reading frame 6 (orf 6) or open reading frame 3 (orf 3) of the E4 coding region; and the VA RNA coding region, are thought to participate in the accessory process. See, e.g., U.S. Pat. Nos. 5,945,335 and 6,004,797, incorporated herein by reference in their entireties; and Janik et al., (1981) Proc. Natl. Acad. Sci. USA 78:1925-1929. Herpesvirus-derived accessory functions have been described. See, e.g., Young et al., (1979) Prog. Med. Virol. 25:113. Vaccinia virus-derived accessory functions have also been described. See, e.g., Carter, B. J. (1990), supra., Schlehofer et al., (1986) Virology 152:110-117.

By “recombinant virus” is meant a virus that has been genetically altered, e.g., by the addition or insertion of a heterologous nucleic acid construct into the particle.

By “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion and both strands are equally infectious.

A “recombinant AAV virion,” or “rAAV virion” is defined herein as an infectious, replication-defective virus composed of an AAV protein shell, encapsidating a heterologous nucleotide sequence of interest which is flanked on both sides by AAV ITRs. A rAAV virion is produced in a suitable host cell which has had an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al., (1981) Gene 13:197. Such techniques can be used to introduce one or more exogenous DNA moieties, such as a nucleotide integration vector and other nucleic acid molecules, into suitable host cells.

The phrase “delivering a gene” or “transferring a gene” refers to methods or systems for reliably inserting foreign DNA into host cells, such as into muscle cells. Such methods can result in transient or long term expression of nonintegrated transferred DNA, extrachromosomal replication and expression of transferred replicons (e.g., episomes), or integration of transferred genetic material into the genomic DNA of recipients. Gene transfer provides a unique approach for the treatment of acquired and inherited diseases. A number of systems have been developed for gene transfer into mammalian cells. See, e.g., U.S. Pat. No. 5,399,346.

The term “host cell” denotes, for example, microorganisms, yeast cells, insect cells, and mammalian cells, that can be, or have been, used as recipients of an AAV helper construct, an AAV vector plasmid, an accessory function vector, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

A “coding sequence” or a sequence which “encodes” a particular protein, is a nucleic acid sequence which is transcribed (in the case of DNA) and translated (in the case of MRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

A “nucleic acid” sequence refers to a DNA or RNA sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term “control elements” refers collectively to promoter sequences, polyadenylation signals, transcription termination sequences, upstream regulatory domains, intron sequences, origins of replication, internal ribosome entry sites (“IRES”), enhancers, and the like, which collectively provide for the replication, transcription and translation of a coding sequence in a recipient cell. Not all of these control elements need always be present so long as the selected coding sequence is capable of being replicated, transcribed and translated in an appropriate host cell. For example, control elements of the present invention include one or more ecdysone-responsive elements (EcREs) to which the ecdysone receptor (EcR) binds when it is present as a heterodimer with a retinoid-X-receptor (RXR).

A “promoter” as used herein is a DNA regulatory region capable of binding RNA polymerase in a mammalian cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

The term “transcriptional promoter region” refers collectively to a region which includes control elements, e.g., EcREs, enhancers, intron sequences, and the like, as well as a promoter sequence which binds RNA polymerase to initiate transcription of a downstream coding sequence. Thus, a transcriptional promoter region according to the present invention may include elements derived from the ecdysone transcriptional promoter region, such as EcREs, but may also include a heterologous promoter responsible for initiating transcription, such as, but not limited to, a heat shock protein promoter. See the discussion below for additional promoters that will find use with the present invention.

“Operably linked” refers to an arrangement of elements wherein the components so described are configured so as to perform their intended function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

A promoter “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest. The expression cassette includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

By “isolated” when referring to a nucleotide sequence, is meant that the indicated molecule is present in the substantial absence of other biological macromolecules such as other nucleotide sequences, chromatin material, etc. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

For the purpose of describing the relative position of nucleotide sequences in a particular nucleic acid molecule throughout the instant application, such as when a particular nucleotide sequence is described as being situated “upstream,” “downstream,” “3′,” or “5′” relative to another sequence, it is to be understood that it is the position of the sequences in the “sense” or “coding” strand of a DNA molecule that is being referred to as is conventional in the art.

The term “therapeutic protein” refers to a protein which is defective or missing from the subject in question, thus resulting in a disease state or disorder in the subject, or to a protein which confers a benefit to the subject in question, such as an antiviral, antibacterial or antitumor function. A therapeutic protein can also be one which modifies any one of a wide variety of biological functions, such as endocrine, immunological and metabolic functions. Representative therapeutic proteins are discussed more fully below.

### II. Modes of Carrying out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

The present invention is directed to novel AAV expression vectors that may be used to introduce genetic material into animals or animal cells for a variety of research and therapeutic uses. A physician or researcher may wish to introduce DNA into an organism (or cells isolated from an organism) for any of several reasons. First, DNA may be introduced to correct a defective gene. Second, DNA may be introduced to specifically delete or mutate a given gene by, for example, homologous recombination. Third, DNA may be introduced to express a protein. Such a protein may be expressed to achieve a therapeutic benefit within the organism treated with rAAV. Alternatively, a protein may be expressed in an organism or in cells isolated from an organism with the goal of isolating and purifying the protein product. Unlike previously described AAV expression vectors, however, the vectors of the present invention permit controlled expression of a heterologous gene.

The present invention allows delivery of therapeutic genes to human tissue and controlled expression of these genes in a highly regulatable manner. Pathological conditions that may benefit from this invention include, but are not limited to, thalassemia, neurodegenerative diseases such as Parkinson's disease, central nervous system injury, vascular disease, single gene defects, and cancer. Thus, the selected therapeutic gene can be one for the treatment of inflammatory diseases, autoimmune, chronic and infectious diseases, including such disorders as cancer, neurological diseases, cardiovascular disease, hypercholestemia; various blood disorders including various anemias, thalasemias and hemophilia; genetic defects such as cystic fibrosis, Gaucher's Disease, adenosine deaminase (ADA) deficiency, emphysema, etc.

AAV containing the CMV regulatory element and the erythropoeitin (Epo) gene has been shown to transduce normal mouse muscle tissue with resulting continual, long-term transgene expression. Kessler et al., (1996) Proc. Natl. Acad. Sci. USA 93:14082-14087. Furthermore, the same vector has been used to demonstrate phenotypic correction in a mouse model of thalassemia. Podsakoff et al., (1998) “Treatment of β-Thalassemic Mice with Intramuscular AAV-Epo.” 1^{st} Annual Meeting of the American Society of Gene Therapy. Seattle, Wash. Abstract; Bohl et al., (2000) Blood 95:2793-2798. Results such as these illustrate the usefulness of AAV as a gene delivery vehicle; however, treatment for many disorders also requires the ability to regulate transgene expression. Regulated gene expression allows clinicians to administer prodrugs to control levels of therapeutic protein, thus optimizing treatment and minimizing potential toxic effects.

The ecdysone-inducible mammalian expression system has been designed to provide tightly-regulated gene expression in a wide variety of mammalian cell types. Development of the system was based on the finding that the insect hormone ecdysone, or its analog ponasterone A (pon A), can activate transcription in mammalian cells harboring both the gene for the Drosophila melanogaster ecdysone receptor and a promoter with upstream regulatory regions containing one or more binding sites for the ecdysone receptor. No et al., (1996) Proc. Natl. Acad. Sci. USA 93:3346-3351. In one embodiment, the present invention is a two-component system comprised of an AAV expression vector and an ecdysone-inducible mammalian expression system. An ecdysone-inducible mammalian expression system for use with the present invention may be designed in part using materials commercially available from, e.g., Invitrogen Corp. (Carlsbad, Calif.) and Stratagene Cloning Systems (La Jolla, Calif.). As outlined in Stratagene's Complete Control™ Inducible Mammalian Expression System instruction manual (La Jolla, Calif., available online at http://www.stratagene.com/manuals/index.shtm), one system design is as follows.

The ecdysone receptor (EcR) is a member of the retinoid-X-receptor (RXR) family of nuclear receptors and is composed of three domains: an N-terminal activation domain (AD), a central DNA-binding domain (DBD), and a C-terminal ligand-binding and dimerization domain (LBD). In insect cells, EcR forms a heterodimer with a second nuclear receptor, ultraspiracle (USP), and together they are bound to co-repressors and tightly repress transcription from the EcR promoter. Chen et al., Proc. Natl. Acad. Sci. USA (1996) 93:7567-7571. When ecdysone is present, it binds to the EcR LBD, the co-repressors are released, co-activators are recruited to the complex, and transcriptional activation occurs.

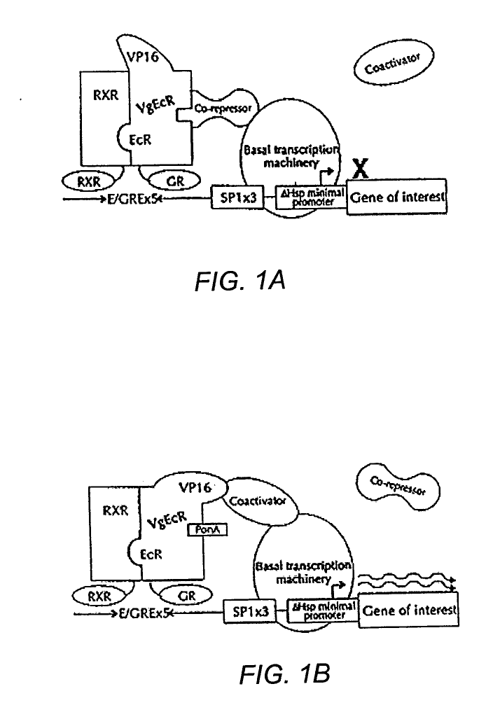

In the ecdysone-inducible mammalian expression system, two separate vectors are employed. The “nuclear receptor” vector expresses the genes for both EcR and RXR (a mammalian homolog of USP) while a second vector contains the transgene of interest under control of a promoter with upstream control elements that include multiple copies of the ecdysone-responsive element (EcRE) found within the EcR promoter region. When both vectors are introduced into mammalian cells, EcR heterodimerizes with RXR, and binds to the EcREs. In the absence of pon A, transcription of the associated transgene is repressed (FIG. 1A). When pon A is present and binds to the receptor, the receptor complex activates transcription of the associated transgene (FIG. 1B).

This inducible mammalian expression system has several advantages over other inducible systems. Pon A has no known measurable effects on mammalian physiology. Pon A has a short in vivo half-life, and its lipophilic nature allows it to efficiently penetrate all tissues, including brain. The result is rapid and potent induction of gene expression and rapid clearance. 1000-fold inductions of reporter genes, with negligible basal expression, have been obtained with this system. Wyborski, D. and Vaillancourt, P., Strategies (1999) 12:1-4 (available online at http://www.stratagene.com/vol12_{—}1/p1-4.htm). Furthermore, both the EcRE recognition sequence and the EcR protein have been modified to avoid pleiotropic interactions with endogenous pathways in mammalian host cells.

The present invention makes use of the above-described system as follows. In particular, the system described herein includes at least two, and sometimes three, AAV vectors that work together such that expression of the gene of interest is tightly regulated. The three separate components of the system include: (1) an AAV vector which bears the transgene of interest operatively linked to a transcriptional promoter region; (2) an AAV nuclear receptor vector that includes the coding sequence for EcR and optionally the coding sequence for RXR; and (3) if desired and not present in the AAV nuclear receptor vector, an AAV vector that includes the coding sequence for RXR. This third component, as well as the presence of the coding sequence for RXR on the AAV nuclear receptor vector, may optionally be excluded, particularly in cases where the system is used in cells that endogenously produce RXR.

### 1. AAV Expression Vectors

AAV expression vectors are constructed using known techniques to at least provide as operatively linked components in the direction of transcription, a transcriptional promoter region which includes a promoter with a transcriptional initiation region, a coding sequence, i.e., a gene encoding the desired transgene such as a therapeutic protein, and in the case of the nuclear receptor vector(s), a gene encoding EcR and optionally a gene encoding RXR; and transcription termination/polyadenylation signals. The control elements are selected to be functional in a mammalian cell. The resulting construct which contains the operatively linked components is bounded (5′ and 3′) with functional AAV ITR sequences. Spacer sequences are optionally present between the polyadenylation sequence and one or both of the flanking ITRs.

In particular, the nucleotide sequences of AAV ITR regions are known. See, e.g., Kotin, R. M., (1994) Human Gene Therapy 5:793-801; Berns, K. I. “Parvoviridae and their Replication” in Fundamental Virology, 2nd Edition, (B. N. Fields and D. M. Knipe, eds.) for the AAV-2 sequence. AAV ITRs used in the vectors of the invention need not be the wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAVX7, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

The transcriptional promoter region includes a promoter which allows RNA polymerase to specifically bind to the DNA sequence in order to initiate transcription. The promoter may be any promoter capable of binding RNA polymerase and directing transcription thereof in a mammalian cell, when operably linked thereto. Typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, a promoter derived from the murine metallothionein gene, and a heat shock (hsp) promoter. Other inducible promoters for use in the transcriptional promoter region include the tetracycline promoter (see, e.g., Bohl et al., (1998) Blood 92:1512-1517; Baron et al., (1999) Proc. Natl. Acad. Sci. USA 96:1013-1018; Rossi et al., (1998) Nature Gen. 20:389-393; Serguera et al., (1999) Hum. Gene Ther. 10:375-383); the rapamycin promoter (see, e.g., Ye et al., (1999) Science 283:88-91; Rivera et al., (1999) Proc. Natl. Acad. Sci. USA 96:8657-8662; Magari et al., (1997) J. Clin. Inv. 100:2865-2872; Liberles, et al., (1997) Proc. Natl. Acad. Sci. USA 94:7825-7830); and the RU486/mifepristone promoter system (Burcin et al. (1998) Proc. Natl. Acad. Sci. USA 96:355-360; Oligino et al., (1998) Gene Ther. 5:491-496; Abruzzese et al., (1999) Hum. Gene Ther. 10:1499-1507). These promoters need not be present in their entireties, but need only retain those elements necessary for directing transcription of a downstream sequence. In fact, due to the size constraints for packaging AAV vectors, minimal promoter sequences are often desirable. These and other promoters can be obtained from commercially available plasmids, using techniques well known in the art. See, e.g., Sambrook et al., supra.

One or more enhancer sequences may also be present in the transcriptional promoter region of the AAV constructs, such as, but not limited to the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 elements derived from human CMV, such as elements included in the CMV intron A sequence as described in Boshart et al., Cell (1985) 41:521, and one or more, preferably at least about three or more SP1 elements, which enhance transcription by interacting with endogenous SP1 transcription factors.

For example, an intron sequence may also be present, located upstream of the gene of interest, in order to enhance expression thereof. Introns are non-coding regions present in most pre-mRNA transcripts produced in the mammalian cell nucleus. Intron sequences can profoundly enhance gene expression when included in heterologous expression vectors. See, e.g., Buchman et al., Molec. Cell. Biol. (1988) 8:4395-4405. Such intron sequences are known and include those derived from, e.g., the human growth hormone sequence, a beta-globin derived intron sequence, a thymidine kinase-derived intron sequence, intron A of the human CMV IE1 enhancer/promoter, and the like.

In the AAV constructs bearing the transgene of interest, the transcriptional promoter region will contain at least one EcRE sequence, preferably at least about three sequences, and most preferably five or more EcRE sequences derived from the regulatory region upstream of the ecdysone promoter in the ecdysone transcriptional promoter region. Such sequences are known in the art (see, e.g., No et al., (1996) Proc. Natl. Acad. Sci. USA 93:3346-3351) and can be derived from commercially available vectors such as those marketed by Invitrogen Corporation (Carlsbad, Calif.).

The coding sequence for the transgene of interest will be less than 5 kilobases (kb) in size and will include, for example, a gene that encodes a protein that is defective or missing from a recipient subject or a gene that encodes a protein having a desired biological or therapeutic effect (e.g., an antibacterial, antiviral or antitumor function). Suitable DNA molecules include, but are not limited to, those encoding proteins used for the treatment of endocrine, metabolic, hematologic, cardiovascular, neurologic, musculoskeletal, urologic, pulmonary and immune disorders, including such disorders as inflammatory diseases, autoimmune, chronic and infectious diseases, such as cancer, hypercholestemia, insulin disorders such as diabetes, growth disorders, various blood disorders including various anemias, thalassemias and hemophilia; genetic defects such as cystic fibrosis, Gaucher's Disease, Hurler's Disease, adenosine deaminase (ADA) deficiency, emphysema, or the like.

To exemplify the invention, the gene encoding human erythropoietin (hEpo) is used as the transgene. Epo is a hormone which controls the production of red blood cells in the bone marrow. The sequence of this gene, as well as methods of obtaining the same, have been described in, e.g., U.S. Pat. No. 4,954,437, incorporated herein by reference in its entirety, as well as in Jacobs et al. (1985) Nature 313:806-810; Lin et al. (1985) Proc. Natl. Acad. Sci. USA 82:7580; International Publication Number WO 85/02610; and European Patent Publication Number 232,034 B1. The recombinant AAV virions described herein which include a gene encoding Epo, or encoding an analog or derivative thereof having the same function, are particularly useful in the treatment of blood disorders characterized by defective red blood cell formation, such as in the treatment of anemia. Increased red blood cell production due to the introduction of the Epo gene can be readily determined by an appropriate indicator, such as by comparing hematocrit measurements pre- and post-treatment.

The nuclear receptor AAV vector will include at least the gene encoding an EcR and optionally a gene encoding an RXR. The coding sequences for EcR and RXR are known in the art (see, e.g., No et al., (1996) Proc. Natl.. Acad. Sci. USA 93:3346-3351) and can be derived from commercially available vectors, such as from Invitrogen Corporation (Carlsbad, Calif.).

Transcription terminator/polyadenylation signals may also be present on the various AAV vectors of the invention, located 3′ to the translation stop codon for the coding sequence. Such sequences include, but are not limited to, those derived from SV40, as described in Sambrook et al., supra, as well as polyadenylation and termination sequences derived from human and bovine growth hormone. Spacer sequences are optionally present between the polyadenylation sequence and one or both of the flanking ITRs. The spacer length is variable and chosen to ensure that the final packaged vector length is smaller than 5.2 kb, and preferably 4.1 to 4.9 kb.

The AAV expression vectors can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,858,351; 5,962,313; 5,846,528; 5,173,414 and 5,139,941; International Publication Nos. WO 92/01070 (published 23 Jan. 1992) and WO 93/03769 (published 4 Mar. 1993); Lebkowski et al., (1988) Molec. Cell. Biol. 8:3988-3996; Vincent et al., (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin, (1994) Human Gene Therapy 5:793-801; Shelling and Smith, (1994) Gene Therapy 1:165-169; and Zhou et al., (1994) J. Exp. Med. 179:1867-1875.

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. For example, ligations can be accomplished in 20 mM Tris-Cl pH 7.5, 10 mM MgCl₂, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226.

Additionally, chimeric genes can be produced synthetically to include AAV ITR sequences arranged 5′ and 3′ of one or more selected nucleic acid sequences. Preferred codons for expression of the chimeric gene sequence in mammalian muscle cells can be used. The complete chimeric sequence is assembled from overlapping oligonucleotides prepared by standard methods. See, e.g., Edge, (1981) Nature 292:756; Nambair et al., (1984) Science 223:1299; Jay et al., (1984) J. Biol. Chem. (1984) 259:6311.

In order to produce rAAV virions, AAV expression vectors are introduced into suitable host cells using known techniques, such as by transfection. A number of transfection techniques are generally known in the art. See, e.g., Graham et al., (1973) Virology, 52:456, Sambrook et al., (1989) Molecular Cloning, a laboratory manual, Cold Spring Harbor Laboratories, New York, Davis et al., (1986) Basic Methods in Molecular Biology, Elsevier, and Chu et al., (1981) Gene 13:197. Particularly suitable transfection methods include calcium phosphate co-precipitation (Graham et al., (1973) Virol. 52:456-467), direct micro-injection into cultured cells (Capecchi, M. R., (1980) Cell 22:479-488), electroporation (Shigekawa et al., (1988) BioTechniques 6:742-751), liposome mediated gene transfer (Mannino et al., (1988) BioTechniques 6:682-690), lipid-mediated transduction (Felgner et al., (1987) Proc. Natl. Acad. Sci. USA 84:7413-7417), and nucleic acid delivery using high-velocity microprojectiles (Klein et al., (1987) Nature 327:70-73).

For the purposes of the invention, suitable host cells for producing rAAV virions include microorganisms, yeast cells, insect cells, and mammalian cells, that can be, or have been, used as recipients of a heterologous DNA molecule. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) are preferred in the practice of the present invention. Particularly, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

### 2. AAV Helper Functions

Host cells containing the above-described AAV expression vectors must be rendered capable of providing AAV helper functions in order to replicate and encapsidate the nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof.

By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., Muzyczka, N., (1992) Current Topics in Microbiol. and Immunol. 158:97-129; and Kotin, R. M., (1994) Human Gene Therapy 5:793-801. Suitable homologues of the AAV rep coding region include the human herpesvirus 6 (HHV-6) rep gene which is also known to mediate AAV-2 DNA replication (Thomson et al. (1994) Virology 204:304-311).

By “AAV cap coding region” is meant the art-recognized region of the AAV genome which encodes the capsid proteins VP 1, VP2, and VP3, or functional homologues thereof. These Cap expression products supply the packaging functions which are collectively required for packaging the viral genome. For a description of the AAV cap coding region, see, e.g., Muzyczka, N. and Kotin, R. M. (supra).

AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves.

These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al., (1989) J. Virol. 63:3822-3828; and McCarty et al., (1991) J. Virol. 65:2936-2945. A number of other vectors have been described which encode Rep and/or Cap expression products. See, e.g., U.S. Pat. Nos. 5,139,941; 6,001,650; and 6,027,931, incorporated herein by reference in their entireties.

Both AAV expression vectors and AAV helper constructs can be constructed to contain one or more optional selectable markers. Suitable markers include genes which confer antibiotic resistance or sensitivity to, impart color to, or change the antigenic characteristics of those cells which have been transfected with a nucleic acid construct containing the selectable marker when the cells are grown in an appropriate selective medium. Several selectable marker genes that are useful in the practice of the invention include the hygromycin B resistance gene (encoding Aminoglycoside phosphotranferase (APH)) that allows selection in mammalian cells by conferring resistance to G418 (available from Sigma, St. Louis, Mo.). Other suitable markers are known to those of skill in the art.

### 3. AAV Accessory Functions

The host cell (or packaging cell) must also be rendered capable of providing nonAAV-derived functions, or “accessory functions,” in order to produce rAAV virions. Accessory functions are nonAAV-derived viral and/or cellular functions upon which AAV is dependent for its replication. Thus, accessory functions include at least those nonAAV proteins and RNAs that are required in AAV replication, including those involved in activation of AAV gene transcription, stage specific AAV mRNA splicing, AAV DNA replication, synthesis of Cap expression products and AAV capsid assembly. Viral-based accessory functions can be derived from any of the known helper viruses.

In particular, accessory functions can be introduced into and then expressed in host cells using methods known to those of skill in the art. Typically, accessory functions are provided by infection of the host cells with an unrelated helper virus. A number of suitable helper viruses are known, including adenoviruses; herpesviruses such as herpes simplex virus types 1 and 2; and vaccinia viruses. Nonviral accessory functions will also find use herein, such as those provided by cell synchronization using any of various known agents. See, e.g., Buller et al., (1981) J. Virol. 40:241-247; McPherson et al., (1985) Virology 147:217-222; Schlehofer et al., (1986) Virology 152:110-117.

Alternatively, accessory functions can be provided using an accessory function vector. Accessory function vectors include nucleotide sequences that provide one or more accessory functions. An accessory function vector is capable of being introduced into a suitable host cell in order to support efficient AAV virion production in the host cell. Accessory function vectors can be in the form of a plasmid, phage, transposon or cosmid. Accessory vectors can also be in the form of one or more linearized DNA or RNA fragments which, when associated with the appropriate control elements and enzymes, can be transcribed or expressed in a host cell to provide accessory functions.

Nucleic acid sequences providing the accessory functions can be obtained from natural sources, such as from the genome of an adenovirus particle, or constructed using recombinant or synthetic methods known in the art. In this regard, adenovirus-derived accessory functions have been widely studied, and a number of adenovirus genes involved in accessory functions have been identified and partially characterized. See, e.g., Carter (1990) “Adeno-Associated Virus Helper Functions,” in CRC Handbook of Parvoviruses, vol. I (P. Tijssen, ed.), and Muzyczka, (1992) Curr. Topics. Microbiol. and Immun. 158:97-129. Specifically, early adenoviral gene regions E1A; the E1B 19 kDa protein coding region; the E2A 72 kDa protein coding region; open reading frame 6 (orf 6) or open reading frame 3 (orf 3) of the E4 coding region; and the VA RNA coding region, are thought to participate in the accessory process. See, e.g., U.S. Pat. Nos. 5,945,335 and 6,004,797, incorporated herein by reference in their entireties; and Janik et al., (1981) Proc. Natl. Acad. Sci. USA 78:1925-1929. Herpesvirus-derived accessory functions have been described. See, e.g., Young et al., (1979) Prog. Med. Virol. 25:113. Vaccinia virus-derived accessory functions have also been described. See, e.g., Carter, B. J., (1990), supra., Schlehofer et al., (1986) Virology 152:110-117.

As a consequence of the infection of the host cell with a helper virus, or transfection of the host cell with an accessory function vector, accessory functions are expressed which transactivate the AAV helper construct to produce AAV Rep and/or Cap proteins. The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions.

Following recombinant AAV replication, rAAV virions can be purified from the host cell using a variety of conventional purification methods, such as CsCl gradients. Further, if infection is employed to express the accessory functions, residual helper virus can be inactivated, using known methods. For example, adenovirus can be inactivated by heating to temperatures of approximately 60° C. for, e.g., 20 minutes or more. This treatment effectively inactivates only the helper virus since AAV is extremely heat stable while the helper adenovirus is heat labile.

The resulting rAAV virions can then be used for gene delivery, such as in gene therapy applications, for the production of transgenic animals, in vaccination, ribozyme and antisense therapy, as well as for the delivery of genes to a variety of cell types.

### 4. In vitro and in vivo Delivery of rAAV Virions

Generally, rAAV virions are introduced into cells using either in vivo or in vitro transduction techniques. If transduced in vitro, the desired recipient cell will be removed from the subject, transduced with rAAV virions and reintroduced into the subject. Alternatively, syngeneic or xenogeneic cells can be used where those cells will not generate an inappropriate immune response in the subject.

Suitable methods for the delivery and introduction of transduced cells into a subject have been described. For example, cells can be transduced in vitro by combining recombinant AAV virions with cells e.g., in appropriate media, and screening for those cells harboring the DNA of interest using conventional techniques such as Southern blots and/or PCR, or by using selectable markers. Transduced cells can then be formulated into pharmaceutical compositions, described more fully below, and the composition introduced into the subject by various techniques, such as by intramuscular, intravenous, subcutaneous and intraperitoneal injection, or by injection into smooth and cardiac muscle, using e.g., a catheter.

For in vivo delivery, the rAAV virions will be formulated into pharmaceutical compositions and will generally be administered parenterally, e.g., by intramuscular injection directly into skeletal or cardiac muscle, or by intravenous, subcutaneous or intraperitoneal injection.

Pharmaceutical compositions will comprise sufficient genetic material to produce a therapeutically effective amount of the protein of interest, i.e., an amount sufficient to reduce or ameliorate symptoms of the disease state in question or an amount sufficient to confer the desired benefit. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

Appropriate doses will depend on the mammal being treated (e.g., human or nonhuman primate or other mammal), age and general condition of the subject to be treated, the severity of the condition being treated, the particular therapeutic protein in question, its mode of administration, among other factors. An appropriate effective amount can be readily determined by one of skill in the art.

Thus, a therapeutically effective amount” will fall in a relatively broad range that can be determined through clinical trials. For example, for in vivo injection, i.e., injection directly to skeletal or cardiac muscle, a therapeutically effective dose will be on the order of from about 10⁶ to 10¹⁵ of the rAAV virions, more preferably 10⁸ to 10¹² rAAV virions. For in vitro transduction, an effective amount of rAAV virions to be delivered to muscle cells will be on the order of 10⁸ to 10¹³ of the rAAV virions. The amount of transduced cells in the pharmaceutical compositions will be from about 10⁴ to 10¹⁰ muscle cells, more preferably 10⁵ to 10⁸ muscle cells. When the transduced cells are introduced to vascular smooth muscle, a lower dose may be appropriate. Other effective dosages can be readily established by one of ordinary skill in the art through routine trials establishing dose response curves.

Dosage treatment may be a single dose schedule or a multiple dose schedule. Moreover, the subject may be administered as many doses as appropriate. One of skill in the art can readily determine an appropriate number of doses.

Ecdysone or an analog thereof capable of binding to the ecdysone receptor, such as but not limited to ponasterone A (pon A), is also administered to the subject in order cause expression of the transgene of interest. Generally, ecdysone, or an analog thereof will be administered in excess to assure that a maximum amount of induction of gene expression occurs. Such an amount is readily determined by one of skill in the art through routine trials and is preferably within the range of 5-500 mg/kg, more preferably about 100 to 400 mg/kg, preferably about 250 mg/kg. Ecdysone or an analog thereof may be delivered prior to, concomitant with, or subsequent to, transduction with the various AAV virions of the invention.

### III. Experimental

Below are examples of specific embodiments for carrying out the present invention. The examples are offered for illustrative purposes only, and are not intended to limit the scope of the present invention in any way. Those of skill in the art will readily appreciate that the invention may be practiced in a variety of ways given the teaching of this disclosure.

Efforts have been made to ensure accuracy with respect to numbers used (e.g., amounts, temperatures, etc.), but some experimental error and deviation should, of course, be allowed for.

### EXAMPLE 1

### Plasmid Construction

One adaptation of the above-described system in order to provide ecdysone-inducible AAV gene therapy is for the treatment of thalassemia. For this application, several vectors were made by the inventors herein that, when used in certain combinations, were capable of pon A-regulated human Epo expression.

### 1A. Construction of pV4.1c hEPO #216

A plasmid containing CMV-driven Epo was generated to serve as a non-regulated expression vector control and to be used as the starting material for generating other plasmid constructs of the invention. Plasmid pV4.1c hEPO #216 was constructed by first generating several intermediate plasmids as follows.

p4.1c: A synthetic DNA encoding the restriction enzyme sites NotI-MluI-Ecl136II-SstII-SfuI-SmaI-SfuI-ClaI-BglII-SnaBI-BstEII-PmlI-RsrII-NotI and having the sequence (CGGCCGCACGCGTGAGCTCCGCGGTTCGAATCCCGGGATTCGAACATCGATA AAAGATCTACGTAGGTAACCACGTGCGGACCGAGCGGCCGC) was cloned into the blunted KasI and EarI(partial) sites of pUC119 (the vector fragment is 2757 bp in length). A 653 bp SpeI(blunted)-SacII(blunted) fragment encoding the CMV immediate early (IE) promoter, and a 488 bp, SmaI-DraIII fragment containing the human growth hormone polyadenylation site, were cloned into the Ecl136II and SnaBI sites of the afore-mentioned plasmid, respectively. A chimeric intron composed of the splice donor from the first intron of CMV IE gene and the splice acceptor from the second intron of the human β-globin gene was then installed into the SmaI site of the plasmid in two steps. A DNA fragment encoding the CMV IE gene first intron splice donor was produced by PCR using isolated CMV DNA (strain ad169) as template and the following primers, GGCCGGGAACGGTGCATT, and GGGCAAGGGGGTGGGCCTATA. This 87 bp fragment was ligated into the SmaI site of the plasmid intermediate. The resulting plasmid was cleaved with BstXI and SmaI, blunted with T4 DNA polymerase, and a 398 bp DraI-EcoRI(blunt) fragment encoding the human β-globin second intron splice acceptor was ligated into the plasmid. The construction of p4.1c was completed by ligation of a polylinker encoding the restriction sites ClaI-EcoRI-SmaI-BamHI-XbaI-SalI-PstI-HinDIII-XhoI-Eco47III-XhoI-BglII between the ClaI and BglII sites of the last intermediate plasmid. The sequence of this synthetic DNA was ATCGATTGAATTCCCCGGGGATCCTCTAGAGTCGACCTGCAGAAGCTTGCTCT CGAGCAGCGCTGCTCGAGAGATCT.

p4.1c mEPO: p4.1c was digested with SmaI and a 2812 bp SmaI(partial)-NcoI(blunted) fragment encoding all of the exons of the mouse erythropoietin gene was inserted. The Kozak sequence around the initiator methionine was changed to the optimally translated sequence, CCACCATG, using oligonucleotide directed mutagenesis. The sequence of the mutagenic oligonucleotide was AGCTAGGCGCCACCATGGGGGTGC.

pV4.1c mEPO: The polylinker and lacZ alpha fragment expression cassette of pUC119 was replaced by a single Sse8387I site by ligation of the following synthetic DNA fragment in the plasmid vector after digestion with AflIII and EheI, GGCGCCCCTGCAGGACATGT. The resulting plasmid was cut with Sse8387I and the 4772 bp Sse8387I fragment from pW1909adhlacZ that contains the ITR-bounded lacZ expression cassette was ligated to it. The resulting plasmid was called intermediate1. Next, p4.1c mEPO was digested with NotI and the 4582 bp fragment encoding the mEPO expression cassette was isolated. One copy of a synthetic DNA fragment that encodes the D region of the AAV ITR was ligated to each end. The sequence of this synthetic fragment was GCGGCCGCAGGAACCCCTAGTGATGGAGTTGG. The product of this reaction was ligated into the 2831 bp, plasmid vector encoding MscI fragment of intermediate1(above) to form pV4.1c mEPO.

p4.1c hEPO: p4.1c was cleaved with SmaI and the 718 bp, PpuMI-NcoI fragment of the human Epo cDNA (blunted) was ligated into this site. The translational initiation sequence was then modified by oligonucleotide -directed mutagenesis using the following mutagenic oligo: catcgattgaattccaccatgggggt. The resulting construct was cleaved with PmlI and the 1765 bp, EcoRV-HincII fragment of the LacZ gene was ligated into it.

Finally, p4.1c hEPO and pV4.1c mEPO were cleaved with Not I. The 4133 bp Not fragment from p4.1c hEPO was ligated into the 3444 bp vector fragment from pV4.1c mEPO to create pV4.1c hEPO #216.

### 1B. Construction of AAV-Ecd1a-hEpo and AAV-Ecd1b-hEpo

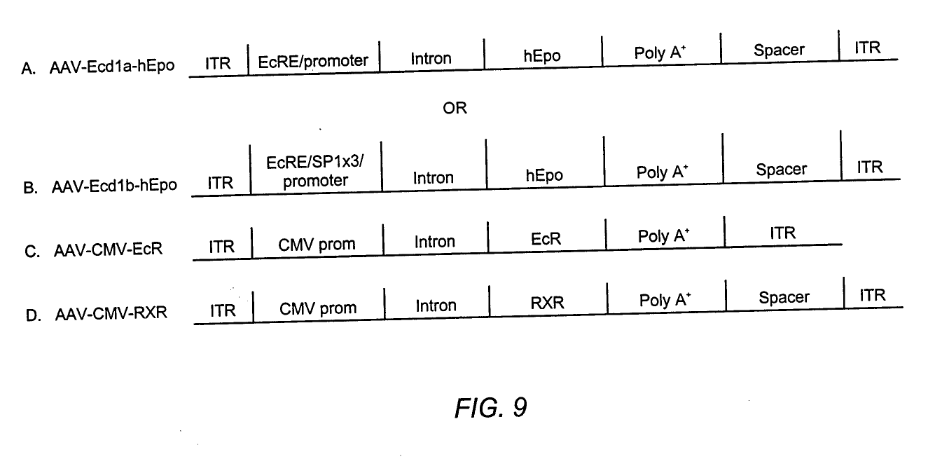

Schematic depictions of AAV-Ecd1a-hEpo and AAV-Ecd1b-hEpo are shown in FIGS. 9A and 9B, respectively. These two vectors are different variations of the same construct. Both contain an ecdysone promoter located upstream from the human Epo gene. The ecdysone promoter in the first vector (referred to as “1a”) contained five copies of the E/GRE recognition sequence located upstream from the ΔHsp minimal promoter. The second (“1b”) contained the same sequence plus three SP1-binding sites located between E/GRE and ΔHsp (as pictured in FIG. 1). SP1 elements enhance expression at the level of transcription by interacting with SP1 transcription factors that are endogenous to mammalian cells. The SP1-containing promoter, 1b, can be induced to absolute expression levels that are five-fold greater than levels obtained with the 1a sequence; however, basal levels of expression are higher as well.

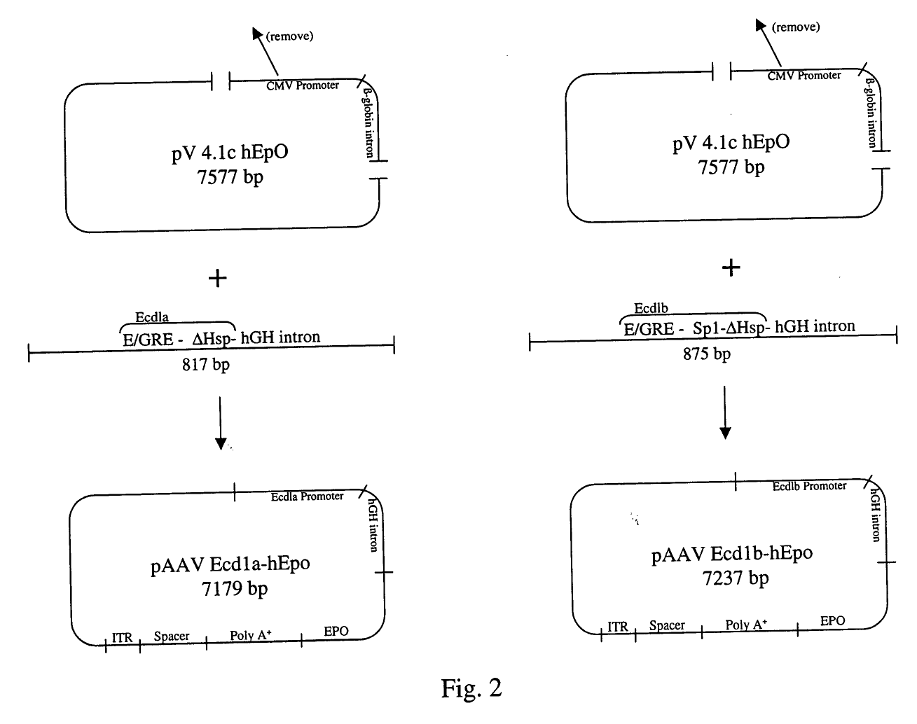

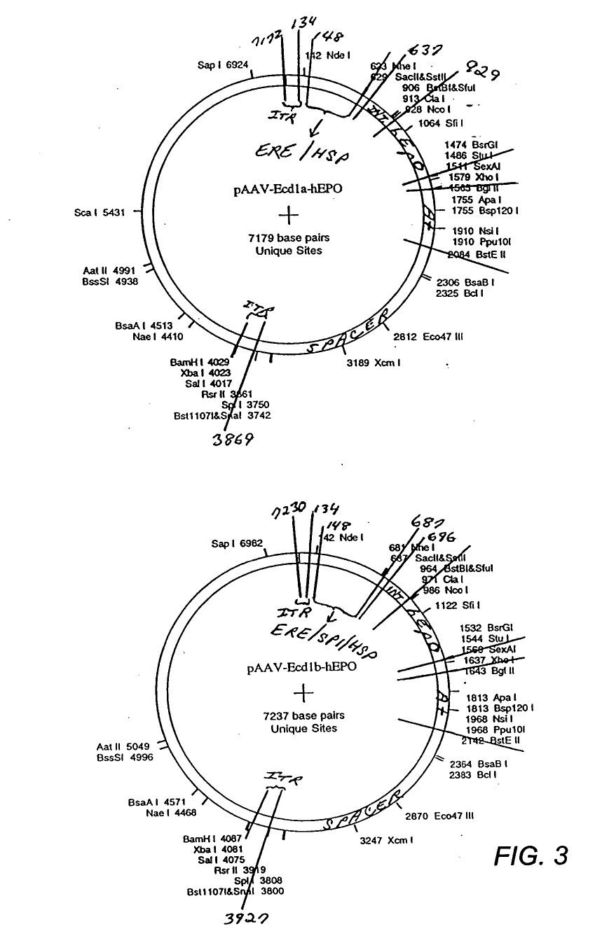

pAAV-Ecd1a-hEpo and pAAV-Ecd1b-hEpo were generated using standard recombinant DNA techniques as shown in FIG. 2. Briefly, the CMV promoter and beta-globin-derived intron sequence were removed from Avigen's pV4.1c hEpo #216 plasmid and replaced with an 817 bp sequence containing Invitrogen's ecdysone promoter (originating from Invitrogen's pIND plasmid) and the human growth hormone intron sequence, or an 875 bp sequence containing Invitrogen's ecdysone-SP1 promoter (originating from Invitrogen's pIND(SP1) plasmid and the human growth hormone intron sequence. The resulting vectors were named pAAV-Ecd1a-hEpo and pAAV-Ecd1-bhEpo, respectively. The final constructs contained an ecdysone promoter (with or without SP1 elements), intron sequence (human growth hormone), the human Epo gene, human growth hormone polyadenylation sequence, “filler” DNA from the β-galactosidase gene (LacZ spacer) and flanking AAV inverted terminal repeats (ITRs) (FIG. 3).

### IC. Construction of pAAV-CMV-EcR

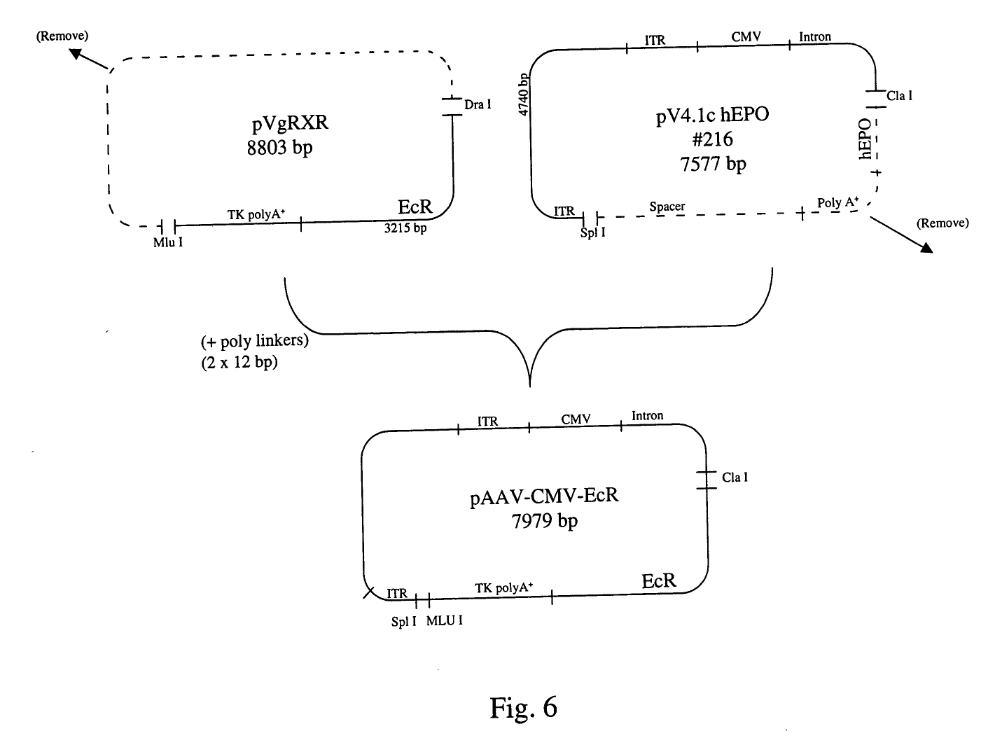

Plasmid pAAV-CMV-EcR was generated using standard recombinant DNA techniques as shown in FIG. 6. Briefly, Invitrogen's EcR gene and attached “poly A” sequence (3215 bp) was used to replace the hEpo-poly A-LacZ spacer sequence in Avigen's pV4.1c hEpo #216 vector. The final construct contains a CMV promoter, intron sequence, the EcR gene, thymidine kinase polyadenylation sequence, and flanking AAV inverted terminal repeats (ITRs). A schematic depiction of pAAV-CMV-EcR is shown in FIG. 9C.

### 1D. Construction of pAAV-CMV-RXR

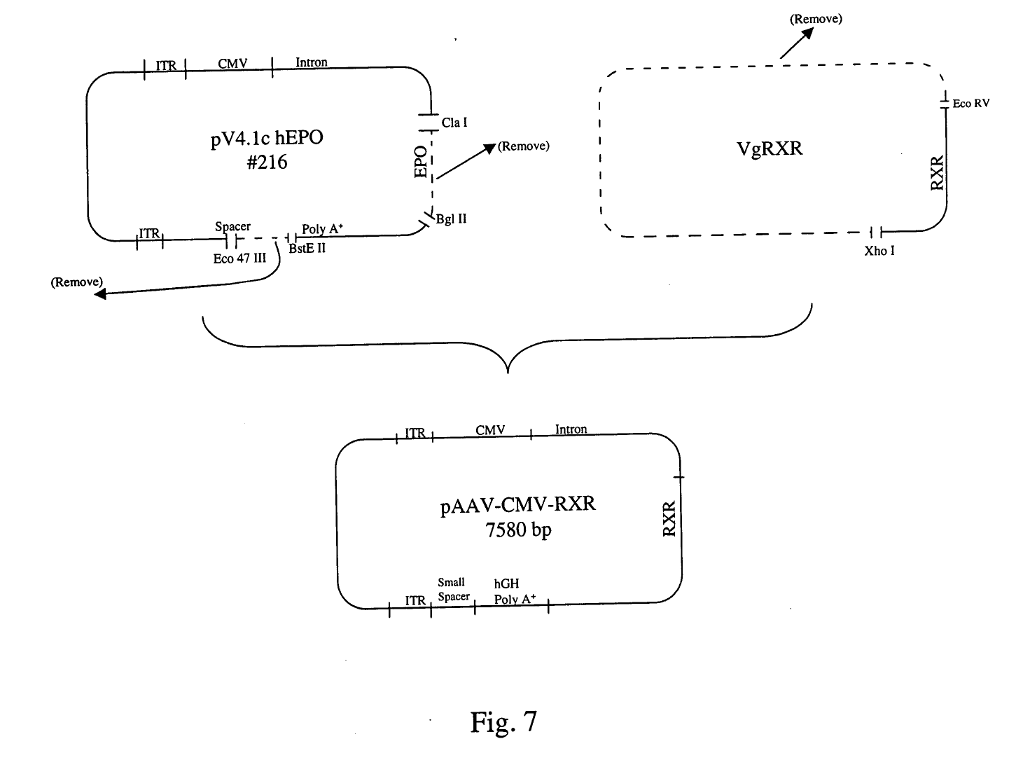

Plasmid pAAV-CMV-RXR was generated using standard recombinant DNA techniques as shown in FIG. 7. Briefly, 728 bp of spacer sequence was removed from Avigen's pV4.1c hEpo #216 vector in order to accommodate the large size of RXR. Next, the RXR gene (Invitrogen, Corp., Carlsbad, Calif.) was used to replace the hEpo sequence in pV4.1c hEpo #216 ss. The final construct contains a CMV promoter, intron sequence, the RXR gene, human growth hormone polyadenylation sequence, LacZ small spacer, and flanking AAV inverted terminal repeats (ITRs). A schematic depiction of pAAV-CMV-RXR is shown in FIG. 9D.

### EXAMPLE 2

### Regulation of AAV Expression Vectors

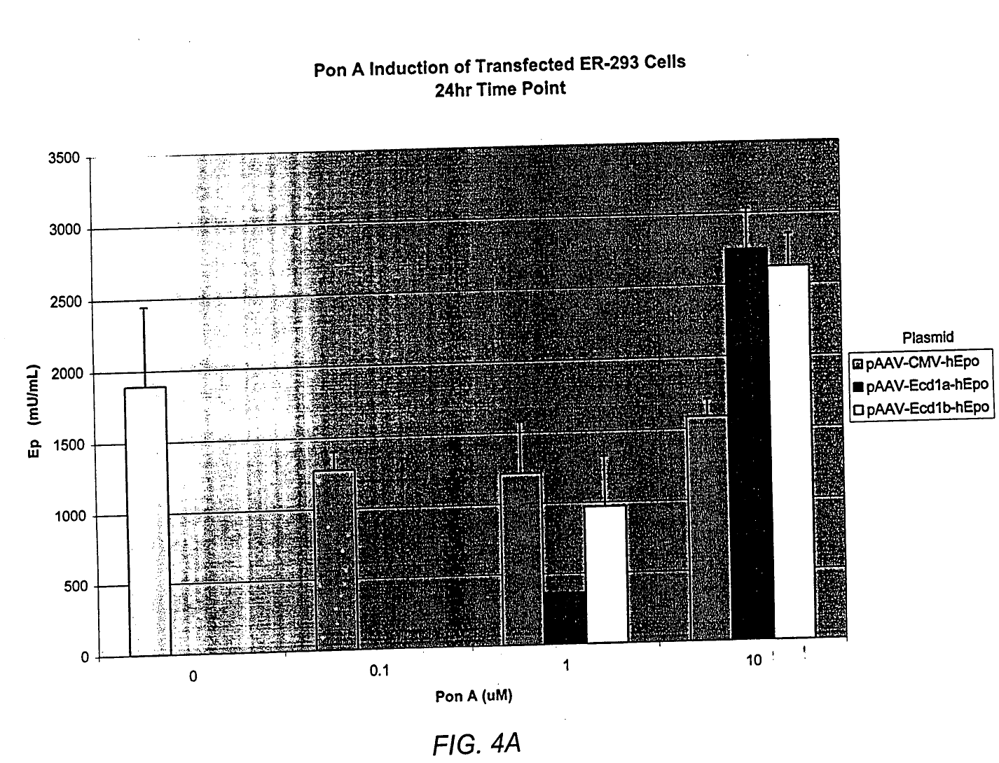

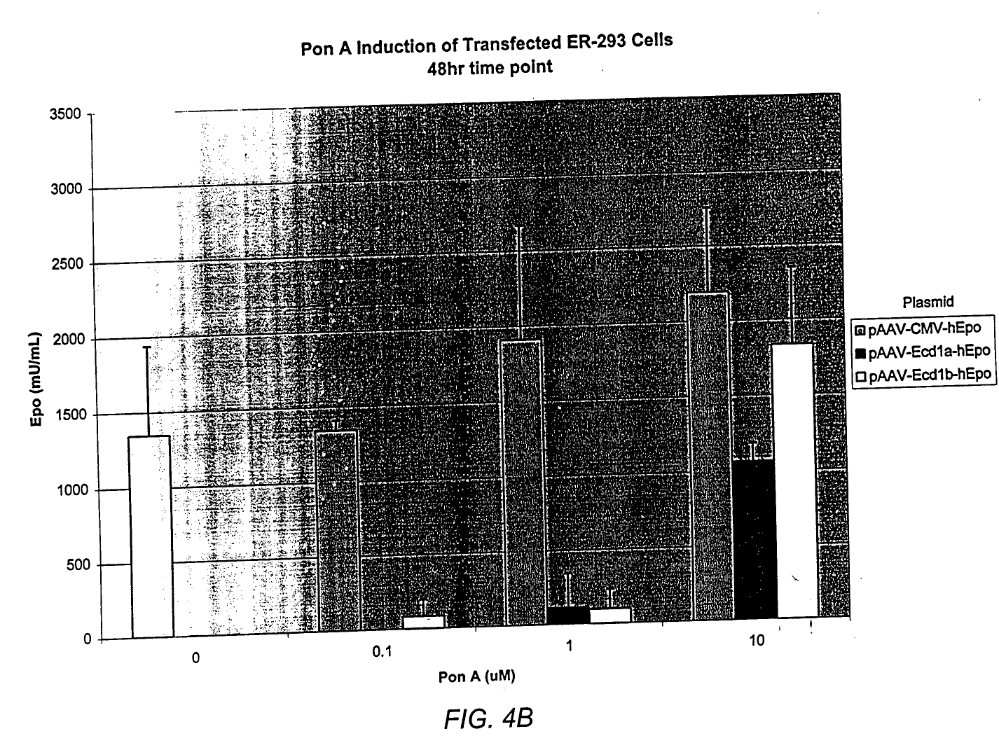

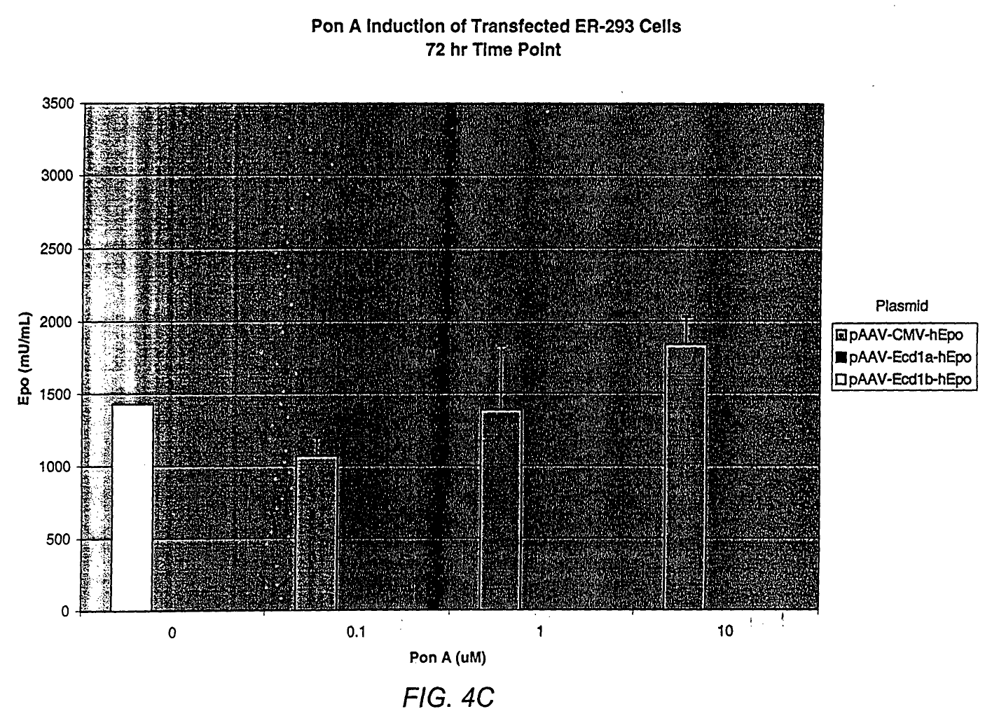

AAV-Ecd1a-hEpo and AAV-Ecd1b-hEpo were transfected into ER-293 cells from Stratagene, La Jolla, Calif. (which have stably integrated EcR and RXR genes) to test their ability to respond to pon A regulation. ER-293 cells grown to 80% confluency in 6-well plates were transfected with 5 μg pAAV-Ecd1a-hEpo, 5 μg pAAV-Ecd1b-hEpo, or 1 μg pAAV-CMV-hEpo and treated with 0, 0.1, 1.0, or 10 μM pon A overnight. Media was collected (and cells were washed) at 24, 48, and 72 hours following the single pon A treatment and analyzed for Epo levels using the human Epo Quantikine IVD kit from R & D Systems (Minneapolis, Minn.), according to manufacturer's recommendations. FIG. 4A illustrates a dose response of pAAV-Ecd1a-hEpo at 24-hours to pon A ranging from undetectable levels of Epo in the absence of pon A to over 2000 mU Epo in the high-dose group. pAAV-Ecd1b-hEpo also demonstrated undetectable basal levels of expression and dose response to pon A treatments. As expected, the Ecd1b construct exhibited greater levels of induction as compared to Ecd1a. A control Epo plasmid with the CMV promoter showed no response to pon A. At the 48 hour timepoint, levels of Epo were lower in the Ecd1a and 1b samples (FIG. 4B) and by 72 hours levels of Epo were negligible (FIG. 4C). These results demonstrate that the AAV-ecdysone system is capable of very highly-controlled gene regulation.

### EXAMPLE 3

### Virion Production and Testing

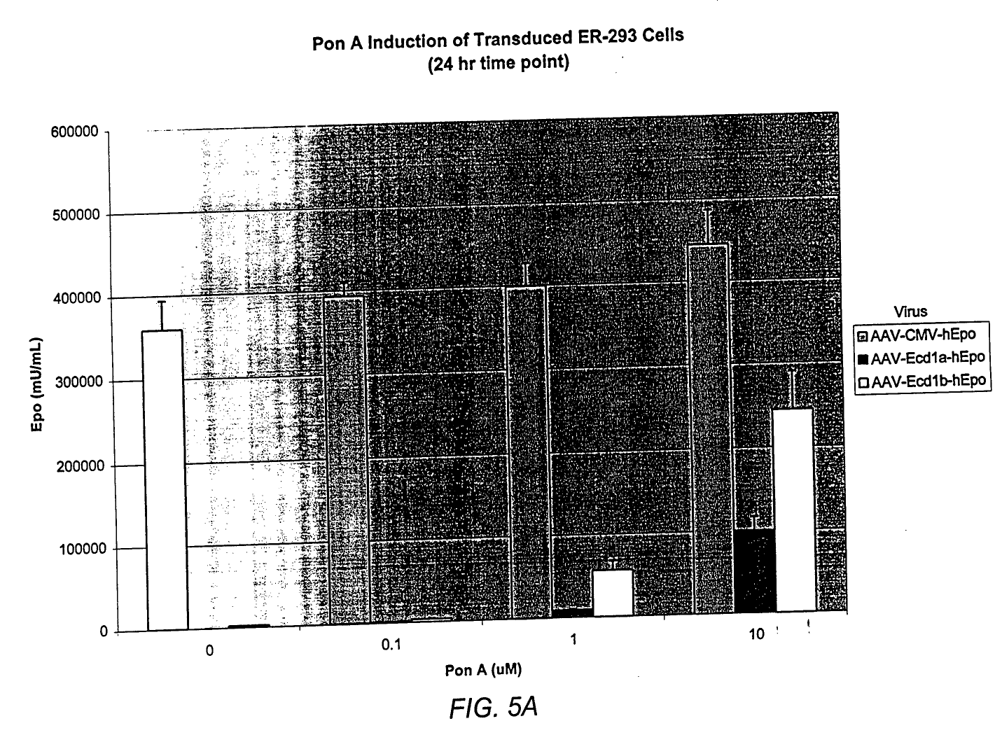

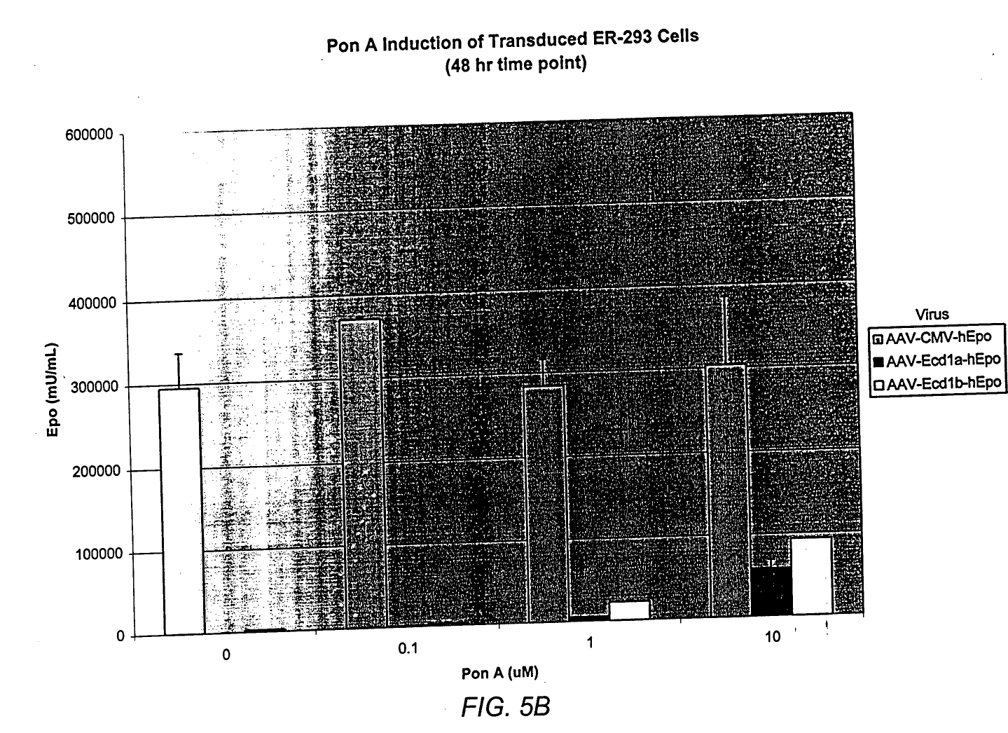

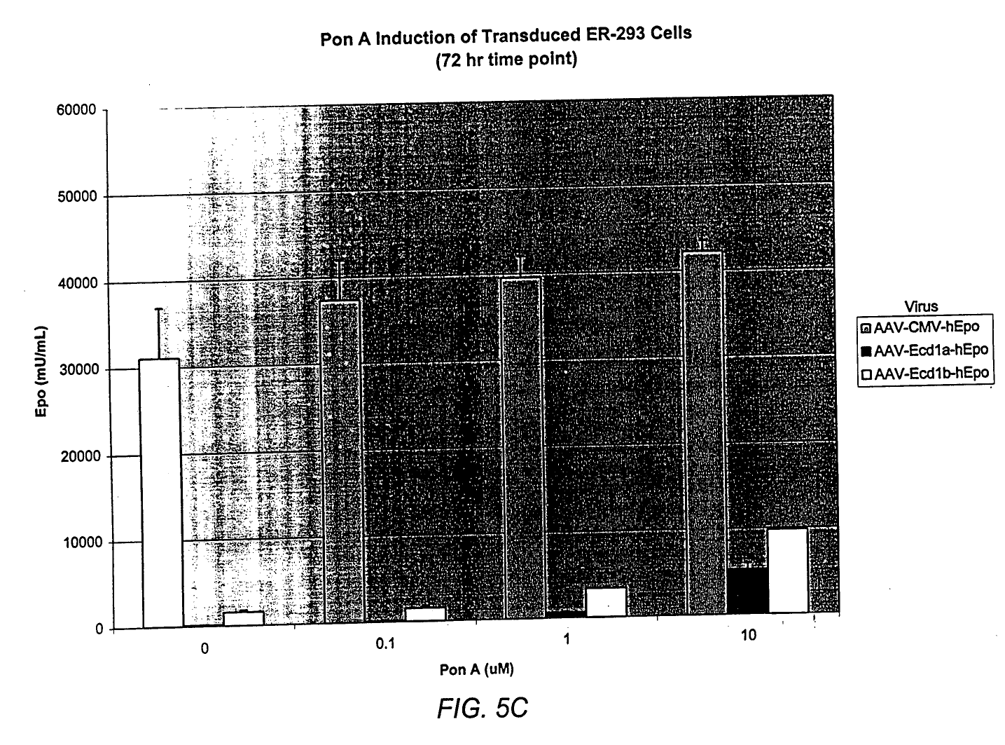

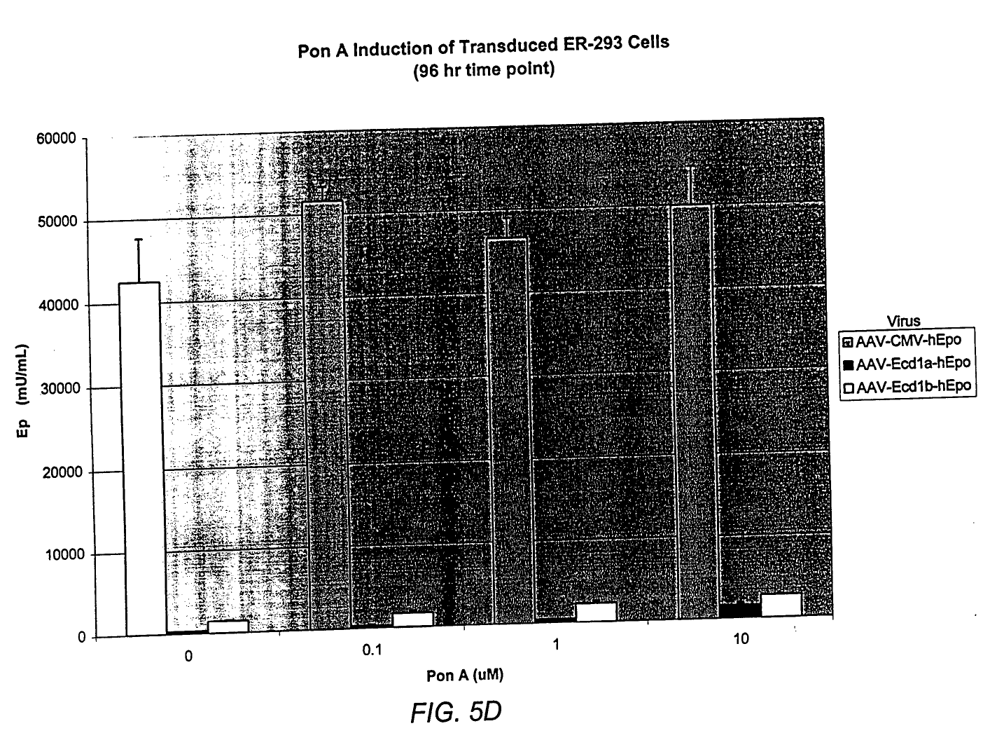

Given these encouraging results, the pAAV-Ecd1a-hEpo and pAAV-Ecd1b-hEpo plasmids were each incorporated into recombinant AAV virions using standard Avigen vector production procedures. See, e.g., Fan et al., (1998) Hum. Gene Ther. 9:2527-2535. These recombinant virions were then used to transduce ER-293 cells to test their ability to respond to pon A regulation. ER-293 cells grown to 80% confluency in 6-well plates were transduced with 1×10¹⁰ particles AAV-Ecd1a-hEpo, 1×10¹⁰ particles AAV-Ecd1b-hEpo, or 2×10⁹ particles AAV-CMV-hEpo and treated with 0, 0.1, 1.0, or 10 μM pon A overnight. Media was collected (and cells were washed) at 24, 48, 72, and 96 hours following the single pon A treatment and analyzed for Epo levels using the human Epo Quantikine IVD kit from R & D Systems (Minneapolis, Minn.) according to manufacturer's recommendations. FIG. 5A illustrates a dose response of AAV-Ecd1a-hEpo at 24-hours to pon A ranging from undetectable levels of Epo in the absence of pon A to over 200,000 mU Epo in the high-dose group. AAV-Ecd1b-hEpo also demonstrated undetectable basal levels of expression and dose response to pon A treatments. As expected, the Ecd1b vector exhibited greater levels of induction than Ecd1a. The control Epo vector with a CMV promoter showed no response to pon A. At the 48 hour timepoint (FIG. 5B), levels of Epo were lower in the Ecd1a and 1b samples. Levels of Epo in these samples continued to drop by 72 hours (FIG. 5C; note change in scale between 48 and 72 hour graphs) and were negligible by 96 hours (FIG. 5D). These results demonstrate that ecdysone-regulated gene therapy vectors are capable of very “tight” gene regulation; that is, basal levels of transgene expression are low or nonexistent, induction is rapid, and withdrawal of pon A results in fast down-regulation of expression.

The nuclear receptor plasmids were also used to generate rAAV virions. In one embodiment, both the EcR and RXR genes are included in the same vector under control of a constitutive promoter (that is, one that is not regulated but “always on” in the target tissue). In another embodiment, endogenous levels of RXR may be high enough that only AAV-Ecd(1a or 1b)-hEpo and a nuclear receptor vector containing EcR (termed AAV-CMV-EcR) are required for gene therapy.

### EXAMPLE 4

### Induction in Muscle Cells

Plasmids pAAV-CMV-RXR, pAAV-CMV-EcR and pAAV-Ecd1a-hEpo (or pAAV-Ecd1b-hEpo) were tested in C2C12 myotubes (mouse muscle cells) to determine if all three components were required for ecdysone-regulated gene therapy in muscle. Mouse C2C12 myoblasts (American Type Culture Collection, Manassas, Va.) were plated in 24-well plates at 75% confluency in growth media (DMEM/10% FCS/Pen-Strep-Glutarnate) and grown for 24 hours to confluency. The media was changed to differentiation media (DMEM/2% horse serum/Pen-Strep-Glutamate) for the following five days. The differentiated myotubes were transduced with either 2 μg pAAV-Ecd1a-hEpo (or pAAV-Ecd1b-hEpo)+0.5 μg pAAV-CMV-EcR (double vector approach) or 2 μg pAAV-Ecd1a-hEpo (or pAAV-Ecd1b-hEpo)+0.5 μg pAAV-CMV-EcR+0.5 μg pAAV-CMV-RXR (triple vector approach), at 2.5×10⁴ MOI for each vector. Samples were then treated with 0, 1.0, or 10 μM pon A (Invitrogen, Corp., Carlsbad, Calif.) overnight. Media was collected at 24, 48, and 72 hours following the single pon A treatment and myotubes were washed three times with PBS following each media collection. Epo concentrations in media samples were measured by ELISA using the human Epo Quantikine IVD kit from R & D Systems (Minneapolis, Minn.) according to manufacturer's recommendations.

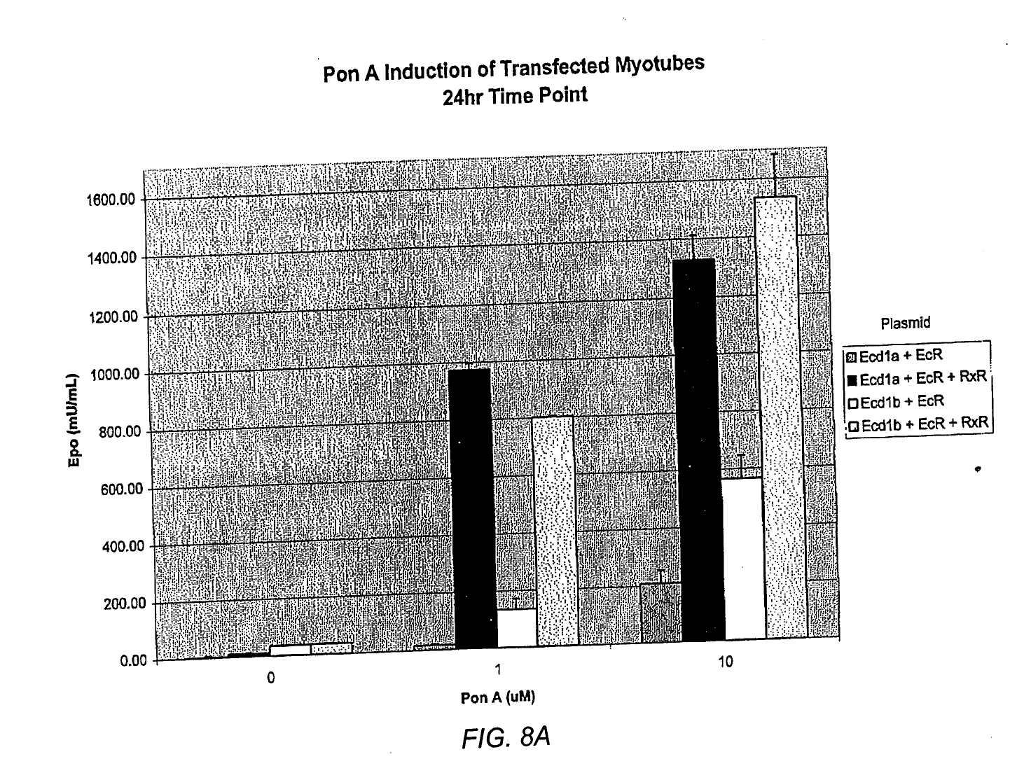

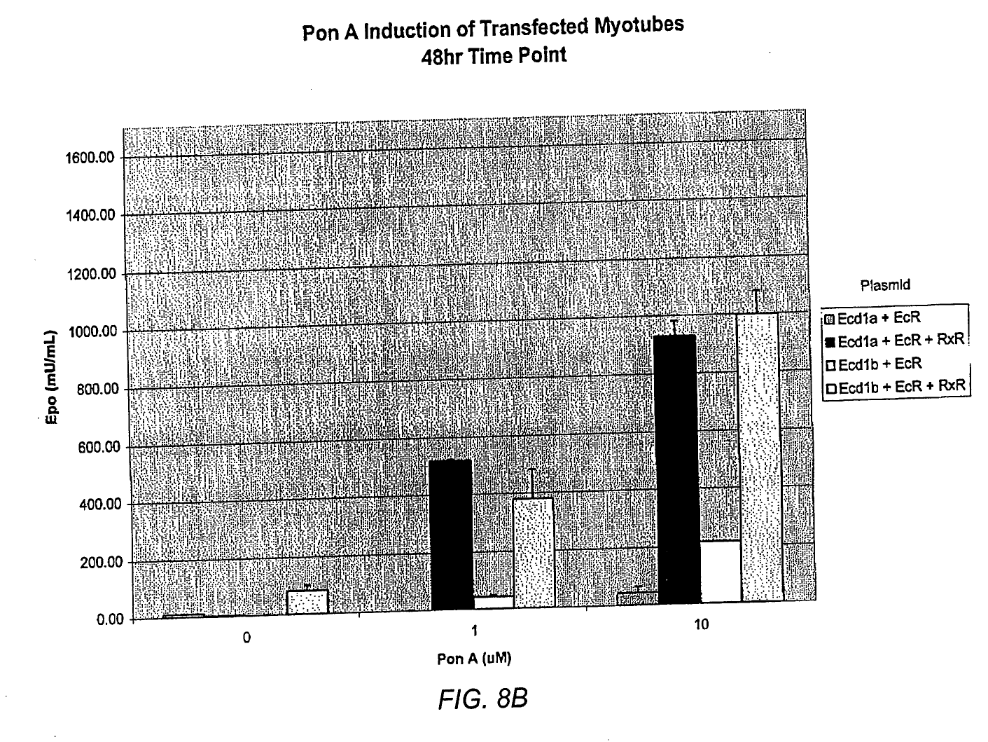

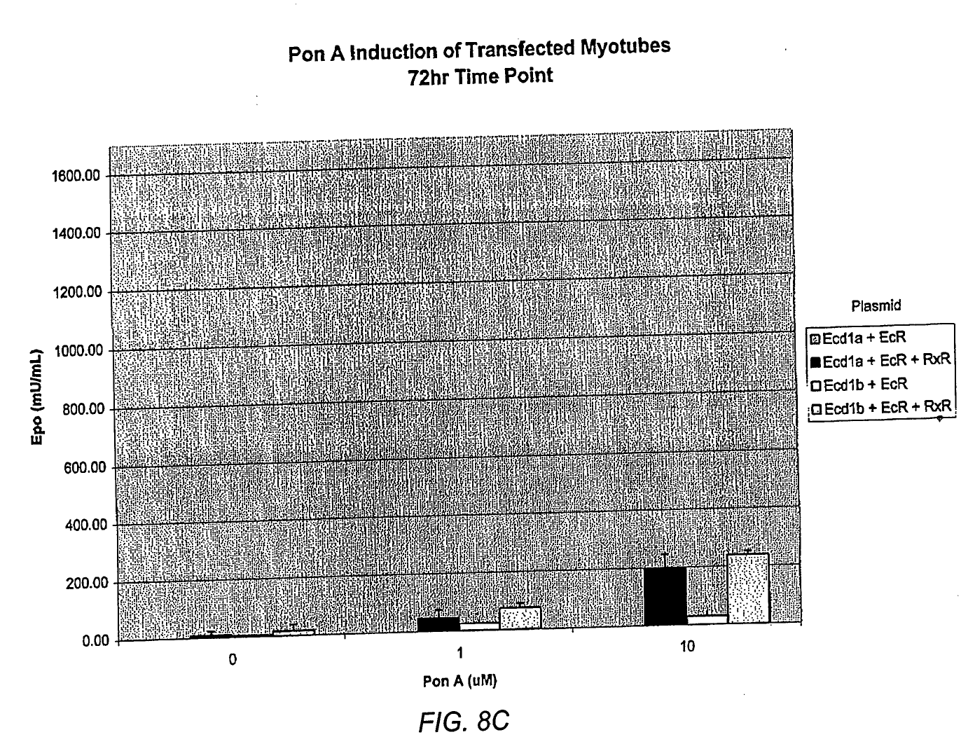

FIGS. 8A-8C illustrate that induction of transgene expression in a muscle-specific cell type is successful using the ecdysone-inducible AAV expression system. Furthermore, while the presence of pAAV-CMV-RXR significantly increases the response, ecdysone-regulated transgenes (pAAV-Ecd1a-hEpo or pAAV-Ecd1b-hEpo) can be effectively induced by co-administering with pAAV-CMV-EcR only (double, rather than triple, vector approach). In particular, FIG. 8A illustrates a dose response of pAAV-Ecd1a-hEpo at 24 hours to pon A ranging from undetectable levels of Epo in the absence of pon A to 1300 mU Epo in the high-dose group when both pAAV-CMV-EcR and pAAV-CMV-RXR are present. In the absence of pAAV-CMV-RXR, levels of Epo reach about 200 mU in the high dose group.

The same experiment using pAAV-Ecd1b-hEpo resulted in production of 1500 mU Epo in the high-dose group at 24 hours when both pAAV-CMV-EcR and pAAV-CMV-RXR were present. In the absence of pAAV-CMV-RXR, levels of Epo reached 560 mU in the high dose group. Thus, the presence of RXR increased Epo expression roughly 6-fold from the Ecd1a promoter and roughly 2.7-fold from the Ecd1b promoter. At the 48 hour timepoint (shown in FIG. 8B), levels of Epo were lower in the Ecd1a and 1b samples whether or not RXR was present. By 72 hours levels of Epo were very low (about 200 mU) when RXR was present and negligible in its absence. See FIG. 8C. These results demonstrate that the AAV-ecdysone system is capable of very highly-controlled gene regulation in muscle both in the presence (triple vector approach) or absence (double vector approach) of pAAV-CMV-RXR.

### EXAMPLE 5

### In Vivo Induction

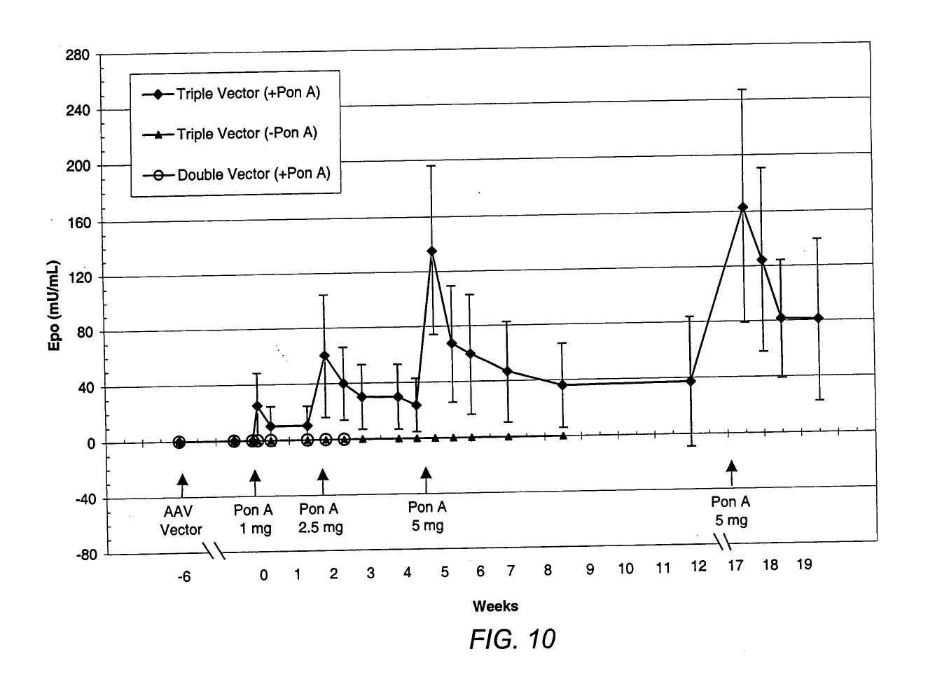

To test the feasibility of ecdysone-regulated gene expression in vivo, the following experiment was conducted. Mice were handled according to National Institutes of Health guidelines. Prior to administration of AAV vectors, 8-week-old female athymic nude mice (Simonson Laboratories, Gilroy, Calif.) received isoflurane anesthesia. Percutaneous injections of vectors into the tibialis anterior muscle of both hindlimbs were performed with the aid of an Instech automated pump at 30 μl/min at the timepoint indicated by the AAV vector arrow in FIG. 10. Double vector-injected mice are represented by open circles and triple vector-injected mice by solid diamonds. Double vector cocktails were composed of AAV-Ecd1a-hEpo and AAV-CMV-EcR (5×10¹⁰ particles each, per mouse, in PBS) and triple vector cocktails were composed of AAV-Ecd1a-hEpo, AAV-CMV-EcR, and AAV-CMV-RXR (5×10¹⁰ particles each, per mouse, in PBS). For delivery, vector cocktails were loaded into a 1 ml plastic syringe (Becton Dickenson) with polypropylene tubing and a 28-gauge needle and injected into muscle at a depth of 2.5 mm. Total volumes per mouse varied from 100-150 μl (50-75 μl/hindlimb). Three and five weeks following transduction, blood was collected to measure hematocrit and basal transgene expression as determined by centrifugation of the blood in a microcapillary tube. At six weeks, mice were induced with tail vein injections of 1 mg ponasterone A (pon A) (Invitrogen) in DMSO. Another group of triple-injected mice were administered DMSO alone (without pon A) and are represented by solid triangles in FIG. 10. Blood was collected from the orbital venus plexus under anesthesia 1, 4, 7, and 14 days later and serum Epo levels were determined by ELISA as described above. Two higher doses of pon A (2.5 mg and 5.0 mg) were similarly tested.

In the absence of inducer, transgene expression was undetectable during the following five weeks. Tail vein injections of DMSO (control) in double or triple vector-injected mice failed to induce production of Epo (FIG. 10). Tail vein injections of pon A in double vector-injected mice also failed to result in detectable levels of serum Epo (FIG. 10). Tail vein injections of 1.0 mg pon A in triple vector-injected mice induced serum Epo levels to a maximum of 25 mU/mL within 24 hours of induction (FIG. 10). Subsequent later injections of 2.5 mg and 5.0 mg pon A demonstrated dose response; serum Epo levels were induced to maximum levels of 60 and 135 mU/mL ,respectively, within 24 hours. Epo levels dropped within one to two weeks following each induction; however, in all of the mice there continued to be a newly established permanently low level of expression. Regulation of the Epo transgene also resulted in modulation of hematocrit. Three weeks following induction with 5 mg pon A, average hematocrit rose in the triple-vector treated mice from 46 to 62 and differed significantly (p<0.05) from control-treated mice. Hematocrits for pon-A treated double vector-injected mice did not differ significantly from controls.

To summarize, ecdysone-regulated expression of the human Epo transgene was demonstrated in-vivo following intramuscular injection of a combination of three AAV vectors. Before exposure to inducer, regulation of the Epo transgene resulted in modulation of hematocrit within a time period consistent with average bone marrow response to hematopoietic stimulants. Triple-vector-injected mice exhibited no detectable levels of hEpo (zero basal expression).

Furthermore, while regulation was obtained in C2C12 cells (mouse myotubes) using only AAV-CMV-EcR and AAV-Ecd1a-hEpo (double vector approach) serum Epo levels were undetectable in pon A-induced double vector-injected mice, suggesting that exogenous RXR may be required for in vivo induction when SP1 sites are not present. Alternatively, double vectors showed a much greater response to inducer in C2C 12 cells when SP1 sites were present, suggesting that RXR may not be required when Ecd1b is employed as opposed to Ecd1a. Ecdysone-regulated expression of hEpo has also been demonstrated for over 60 days in immunocompetent mice.

These results suggest that ecdysone-regulated transgene expression is a viable option for treatment of β-thalassemia and could potentially provide controlled gene therapy for a myriad of AAV applications including hematologic, metabolic, central nervous system, vascular disorders, cancer and other pathological states.

Although preferred embodiments of the subject invention have been described in some detail, it is understood that obvious variations can be made without departing from the spirit and the scope of the invention as defined by the appended claims.

We claim

1. A recombinant adeno-associated virus (AAV) virion comprising a nucleic acid molecule, said nucleic acid molecule comprising a transcriptional promoter region operably linked to a polynucleotide of interest, wherein the transcriptional promoter region comprises at least one ecdysone-responsive element (EcRE), and a promoter capable of directing the in vivo transcription of said polynucleotide of interest in a mammalian cell, located downstream of the at least one EcRE.

2. The recombinant AAV virion of claim 1, wherein the transcriptional promoter region comprises at least five EcREs.

3. The recombinant AAV virion of claim 1, wherein the promoter is a heat shock protein (Hsp) promoter sequence.

4. The recombinant AAV virion of claim 1, wherein the transcriptional promoter region further comprises at least one enhancer sequence.

5. The recombinant AAV virion of claim 4, wherein the enhancer sequence is an SP1 enhancer sequence.

6. The recombinant AAV virion of claim 4, wherein the transcriptional promoter region comprises three SP1 enhancer sequences.

7. A recombinant adeno-associated virus (AAV) virion comprising a nucleic acid molecule, said nucleic acid molecule comprising a transcriptional promoter region operably linked to a polynucleotide of interest, wherein the transcriptional promoter region comprises five ecdysone-responsive elements positioned upstream of a heat shock protein (Hsp) promoter sequence, wherein the transcriptional promoter region is capable of directing the in vivo transcription of said polynucleotide of interest in a mammalian cell.

8. The recombinant AAV virion of claim 7, wherein the transcriptional promoter region further comprises at least one enhancer sequence.

9. The recombinant AAV virion of claim 8, wherein the enhancer sequence is an SP1 enhancer sequence.

10. The recombinant AAV virion of claim 8, wherein the transcriptional promoter region comprises three SP1 enhancer sequences.

11. A recombinant adeno-associated virus (AAV) virion comprising a nucleic acid molecule, said nucleic acid molecule comprising a first coding sequence encoding an ecdysone receptor (EcR) operably linked to control elements capable of directing the in vivo transcription of said first coding sequence in a mammalian cell.

12. The recombinant AAV virion of claim 11, wherein the nucleic acid molecule further comprises a second coding sequence encoding a retinoid-X-receptor (RXR) operably linked to control elements capable of directing the in vivo transcription of said second coding sequence in a mammalian cell.

13. The recombinant AAV virion of claim 12, wherein said first coding sequence and said second coding sequence are present in the same expression cassette.

14. The recombinant AAV virion of claim 12, wherein said first coding sequence and said second coding sequence are present in separate expression cassettes.

15. A recombinant adeno-associated virus (AAV) virion comprising a nucleic acid molecule, said nucleic acid molecule comprising first and second coding sequences, wherein said first coding sequence encodes an ecdysone receptor (EcR) and said second coding sequence encodes a retinoid-X-receptor (RXR), wherein said first and second coding sequences are operably linked to control elements capable of directing the in vivo transcription thereof in a mammalian cell.

16. The recombinant AAV virion of claim 15, wherein said first coding sequence and said second coding sequence are present in the same expression cassette.

17. The recombinant AAV virion of claim 15, wherein said first coding sequence and said second coding sequence are present in separate expression cassettes.

18. A recombinant adeno-associated virus (AAV) virion comprising a nucleic acid molecule, said AAV vector comprising a coding sequence encoding a retinoid-X-receptor (RXR) operably linked to control elements capable of directing the in vivo transcription of said coding sequence in a mammalian cell.

19. A method of producing recombinant adeno-associated virus (AAV) virions comprising:
(a) transfecting a host cell with (i) an AAV vector comprising a transcriptional promoter region operably linked to a polynucleotide of interest, wherein the transcriptional promoter region comprises at least one ecdysone-responsive element (EcRE), and a promoter capable of directing the in vivo transcription of said polynucleotide of interest in a mammalian cell, located downstream of the at least one EcRE; (ii) AAV helper functions; and (iii) AAV accessory functions, wherein said transfecting is done under conditions that allow the formation of recombinant AAV virions; and
(b) purifying the recombinant AAV virions from the host cell.

20. A method of producing recombinant adeno-associated virus (AAV) virions comprising:
